(19) United States
(12) Patent Application Publication
(10) Pub. No.: US 2001/0051760 A1
(43) Pub. Date: Dec. 13, 2001

(54) Use of photofrin as a radiosensitizer

(76) Inventors: Alfons Hofstetter, Unterhaching (DE); Eckhart Duhmke, Tutzing (DE); Moshe Schaffer, Munchen (DE); Giulio Jori, Padua (IT); Luigi Corti, Padua (IT)

Correspondence Address: WEINGARTEN, SCHURGIN, GAGNEBIN, & HAYES, LLP, TEN POST OFFICE SQUARE, BOSTON, MA, 02109, US

(21) Appl. No.: 09/843,977

(22) Filed: Apr. 27, 2001

(30) Foreign Application Priority Data
Apr. 28, 2000 (DE) 100 20 935.1

(51) Int. Cl. A61N 5/02

(52) U.S. Cl. 600/2

(57) ABSTRACT

The invention relates to the use of photofrin as a radiosensitizer in the treatment of tumorous tissue, wherein after a predetermined time interval after application of the photofrin an irradiation of the tumorous tissue with ionizing radiation is effected.

The invention relates to the use of photofrin as a radiosensitizer in the treatment of tumorous tissue.

For treatment of malignant neoplasias often a photodynamic therapy (PDT) is employed. The PDT includes the administration of a photosensitizing substance and the activation thereof with light of suitable wavelength, resulting in mortification of the photosensitized cells. Among other things, in the PDT also porphyrins and their derivatives are employed as photosensitizing substances.

In WO 94/17797, for example, the use of photofrin as a photoactivable substance in the medicinal treatment of a proliferating joint disease of a patient is described, wherein the photofrin accumulates in the joint interior skin tissue. The irradiation of the joint interior skin tissue with light of a photoactivating wavelength then causes the destruction of the tissue. A fraction of hematoporphyrin derivatives (HPD), purified by gel exclusion chromatography, is referred to as “photofrin” or “photofrin III”, respectively, which is responsible for the photosensitizing characteristics. Therein, hematoporphyrin derivatives are to be taken as a chemical preparation of a complex porphyrin mixture that selectively accumulates in tumor tissues. Up to now, the exact composition of the photofrin is not known, hereto see also C. J. Byrne, L. V. Marshallsay and A. D. Ward (1990) “The composition of photofrin” Journal of Photochemistry and Photobiology 6, pages 13-27. However, the PDT described in WO 94/17797 is disadvantageous in that the irradiation and thus the destruction of the affected tissue can only be effected if accessing this tissue is possible and the tissue to be treated can be irradiated directly with light of the appropriate wavelength. However, in numerous tumor diseases this is not the case.

In U.S. No. 5,257,970, a PDT method is described, by which an attempt is made to overcome this disadvantage. Therein, in addition to the photosensitizer, liposomes are injected into a human or an animal, which are heat-sensitive and include separate components of an activating system for the PDT. Thereafter, the liposomes are selectively destroyed at a tumor by direct heat producing systems, thereby releasing and mixing the components of the activating system. Thereby light emission and simultaneous absorption by the photosensitizers is achieved. Alternatively, also another energy transfer to the previously injected photosensitizers or a conversion to a cytotoxic species that may interact with the tumor can be achieved. As a result, the tumor is destroyed. However, as a whole, this method seems to be extremely awkward and in many cases unusable.

In the medical literature there can already be found indications that porphyrins can also be used as radiosensitizers, wherein the radiation required thereto is ionizing radiation such as X radiation, that penetrates the body without difficulty, and therefore must not be guided into close proximity of the tissue to be irradiated by an additional guide member. For example, a marked increase in the tumor radiosensitivity is caused at rabdomyosarcoma by the use of hematoporphyrins (L. Choen and S. Schwartz (1966) “Modification of radiosensitivity by porphyrin II transplanted rabdomyosarcoma in mice” Cancer Research 26, Part 1, Pages 11769-1773; S. Schwartz, M. Krepios, J. Modelevsky, H. Freyholtz, R. Walters and L. Larson (1978) “Modification of radiosensitivity by porphyrins: studies of tumors and other systems” in Diagnosis and Therapy of Porphyrias and Lead Intoxication (Ed. M. Doss) Springer Verlag, Berlin Heidelberg New York, Pages 227-235). However, dependencies of effect on substance dose exhibited that with high hematoporphyrin dose a radioprotection may also occur.

However, such a radioprotection makes an appropriate dosage extremely difficult, since the concentration within the tumor tissue is highly dependent on the state of the tumor to be treated and therefore is difficult to estimate. This results in this kind of therapy in some cases remaining ineffective because of the occurring radioprotection.

Therefore, the object of the present invention is to provide a comparatively simple possibility of being able to effectively treat also tumorous tissue that cannot be irradiated with light due to its position.

This object is achieved by the use of photofrin as a radiosensitizer in the treatment of tumorous tissue, wherein after a predetermined time interval after application of the photofrin an irradiation of the tumorous tissue with ionizing radiation is effected.

As surprisingly exhibited, by a combination of photofrin application and irradiation an inhibition of the tumor growth can be achieved. With none of the photofrin dosages tested up to now a radioprotection by photofrin has been detected. As a result, the components of the hematoporphyrin that cause the radioprotection are most likely to be separated in the preparation of the photofrin. Due to this, photofrin has extraordinary advantages in the tumor treatment in contrast to the previously known radiosensitizers. Photofrin is an extremely selective radiosensitizer because it accumulates in the tumor tissue, and thus the photofrin concentration in the tumor tissue is substantially higher than in the remaining tissues. By the use of ionizing radiation, also inaccessible tumor tissue can be treated successfully and be inhibited in its growth. In addition to the photofrin application and the irradiation with ionizing rays a hyperthermia therapy can also be performed, that causes a synergistic effect.

Additionally labeling the photofrin with a neutron absorber is especially advantageous. Gadolinium has such a neutron capturing effect. The use of gadolinium-labeled photofrin allows not only an extremely effective therapy by neutron radiation, but also a precise diagnosis by nuclear spin resonance. In this manner, the tumor position and the tumor shape can be exactly determined, thereby enabling an extremely aimed treatment of the tumor. Photofrin can also be labeled with boron to be able to perform a conventional boron neutron capture therapy (BNCT). The boron-labeled photofrin accumulates in tumorous tissues like photofrin alone or also gadolinium-labeled photofrin. This therapy proves to be extremely favorable in brain tumors.

The used ionizing radiation can be photon and/or electron and/or gamma and/or alpha and/or beta and/or neutron and/or proton radiation. Therein, several therapy forms or irradiation methods can also simultaneously be selected, respectively, such as interstitial brachytherapy, stereotactic irradiation, gamma knife radiosurgery, and exterior and percutaneous irradiation, respectively. While the physical effect of the porphyrins as photosensitizers is achieved by absorption of light in a wavelength range of 350-900 nm, at present two theoretical approaches for explaining the effective mechanism of the porphyrins as radiosensitizers are discussed. Firstly, the radiosensitivity might be achieved by an increased oxygen concentration in the tumor tissue, which is caused by the high porphyrin concentration in the tumor tissue and the thus improved blood circulation of the tissue. Further, the irradiation with ionizing radiation might result in generation of free radicals that on their part activate porphyrins and thus initiate a series of enzymatic reactions with the result of tumor destruction.

The irradiation can be effected with a radiation dose of 2 to 20 Gy, more particularly 5 to 15 Gy.

In order to prevent a regeneration of the tumor cells after an irradiation, a fractionating irradiation can also be performed. Therein, different irradiation types can also be combined. The photofrin accumulated in the tumor tissue remains in the tumor tissue up to 72 h after the photofrin application. Therefore, a fractionating irradiation is very effective in appropriate time intervals between the individual irradiations, such as 12 h to 24 h. In order to avoid fast regeneration processes, shorter time intervals of 3 h to 12 h, more particularly 4 h to 6 h, can also be selected.

In particularly advantageous manner, the application of the photofrin is performed intravenously. In this manner, an extremely fast distribution of the photofrin in the organism is achieved, which then results in the accumulation of the photofrin in the tumor tissue.

The photofrin is particularly effective if it is injected intravenously in a dose of 7,5 mg to 15 mg per kg body weight.

Further, it seems to be extremely convenient if the time interval between the application of the photofrin and the irradiation is between 12 h and 48 h, however especially 24 h. Within this time interval a sufficient accumulation of the photofrin in the tumor tissue occurs. A time interval of less than 12 h can be selected to achieve an inhibition of the tumor growth also with lower photofrin concentrations in the tumor tissue.

The photofrin can also be a component of a medicine for treatment of tumorous tissue. In this manner, the effect of the photofrin can be combined with favorable characteristics of other substances. Therein, the tumorous tissue can be both human and animal tissue.

Further advantages of the invention will become apparent from the tests stated below.

In a first test female mice each having a body weight of 20-22 g were used as animal models. A Lewis carcinoma was transplanted subcutaneously to each of these mice. In untreated mice the transplanted tumor grew extremely fast and increased its volume twelve-fold within six days. The tumor volume was daily measured with calipers or a micrometer screw, respectively.

Photofrin was injected into the tail vein in a therapeutic dose of 0,5 or 5 mg per kg body weight. The irradiation was effected with 3 Gy 24 h after the intravenous injection of the photofrin. The used 30 mice were divided into five different groups of the same size. The mice of group A were neither treated with photofrin nor irradiated. The mice of group B were exclusively irradiated with 3 Gy. The mice of group C were each injected with 5 mg photofrin per kg body weight without any irradiation following. The mice of group D were irradiated with 3 Gy after injection of photofrin of 5 mg per kg body weight. The mice of group E were also irradiated with 3 Gy after injection of 0,5 mg photofrin per kg body weight.

TABLE 1

| | Tumor size (cm³) | | | | |
|---|---|---|---|---|---|
| Day | A | B | C | D | E |
| 0 | 0.09 | 0.11 | 0.09 | 0.12 | 0.06 |
| 1 | 0.11 | 0.17 | 0.13 | 0.14 | 0.12 |
| 2 | 0.17 | 0.24 | 0.18 | 0.17 | 0.18 |
| 3 | 0.26 | 0.37 | 0.29 | 0.21 | 0.33 |
| 4 | 0.44 | 0.61 | 0.50 | 0.30 | 0.65 |
| 5 | 0.67 | 0.90 | 0.69 | 0.49 | 0.86 |
| 6 | 1.20 | 1.29 | 1.15 | 0.65 | 1.20 |

### Explanation of Symbols

A=control, not injected and not irradiated mice

B=control, irradiated, but not injected mice

C=mice injected with photofrin (5 mg/kg), but not irradiated

D=mice injected with photofrin (5 mg/kg), and irradiated (3 Gy)

E=mice injected with photofrin (0,5 mg/kg), and irradiated (3 Gy)

The above listed data represent the average of the measured values of the respective groups. The average deviation factor did not exceed 0,03 in any of the experiments.

As is apparent from table 1, only the combination of a higher photofrin dose of 5 mg per kg body weight and irradiation has a crucial effect on the tumor growth. Only in the group D of mice, the tumor growth rate has been gradually reduced. After six days, the tumor volume corresponded to only 50% of the average tumor volume of the group A of mice serving as a control. This result is extremely significant and definitely proves that photofrin acts as a radiosensitizer.

In a further test, naked mice having a body weight of 20-22 g and a subcutaneously transplanted tumor “human bladder cancer RT4” were used as animal models. The tumor volume that also in this test was daily measured increased within eleven days by about 3,5 to 3,8 MM³. Photofrin was injected into the tail vein in a therapeutic dose of 5 mg per kg body weight or in a dose of 10 mg per kg body weight. The irradiation was effected with 5 Gy 24 h after the intravenous injection of the photofrin. Five mice each constituted a test group. The mice of group A were neither treated with photofrin nor irradiated. The mice of group B were exclusively irradiated with 5 Gy. The mice of group C were injected with 10 mg photofrin per kg body weight without any irradiation following. The mice of group D were irradiated with 5 Gy after injection of photofrin of 5 mg per kg body weight. The mice of group E were also irradiated with 5 Gy after injection of 10 mg photofrin per kg body weight. After the sixth day, the group B consisted only of four mice, since one mouse died.

The results of the test are listed in table 2.

TABLE 2

| | Tumor size (mm³) | | | | |
|---|---|---|---|---|---|
| Day | A | B | C | D | E |

| | | | | | |
|---|---|---|---|---|---|
| 0 | 3.42 | 3.46 | 3.42 | 3.46 | 3.64 |
| 1 | 3.46 | 3.54 | 3.66 | 3.5 | 3.72 |
| 2 | 3.72 | 3.64 | 3.84 | 3.58 | 3.82 |
| 3 | 4.02 | 3.86 | 4.12 | 3.72 | 3.92 |
| 4 | 4.46 | 4.34 | 4.78 | 3.92 | 4.02 |
| 5 | 5.14 | 5.2 | 5.7 | 4.18 | 4.22 |
| 6 | 5.98 | 5.65 | 6.5 | 4.58 | 4.36 |
| 7 | 7.0 | 6.7 | 8.24 | 5.08 | 4.6 |
| 8 | 8.04 | 7.49 | 9.62 | 5.52 | 4.78 |
| 9 | 9.42 | 8.67 | 11.0 | 5.98 | 5.16 |
| 10 | 10.74 | 10.9 | 12.64 | 6.54 | 5.5 |
| 11 | 12.96 | 13.63 | 14.27 | 7.5 | 5.9 |

### Explanation of Symbols

A=control, not injected and not irradiated mice

B=control, irradiated (5 Gy), but not injected mice

C=mice injected with photofrin (10 mg/kg)

D=mice injected with photofrin (5 mg/kg), and irradiated (5 Gy)

E=mice injected with photofrin (10 mg/kg), and irradiated (5 Gy)

With an irradiation with 5 Gy (group B) an inhibition of the tumor growth until the ninth day could be detected, while subsequently similar growth rates as in the control group A could be observed. Photofrin alone had no influence on the tumor growth compared to the control group (see group C). The results of the groups D and E indicate that by injection of photofrin the tumor growth was gradually retarded. Totally, the tumor volume in the control groups at the 11th day was 1,8times the tumor volume of the group D and 2,3times the tumor volume of the group E. Thus, the higher photofrin dose of 10 mg per kg body weight was more efficient (see group E).

In a third test, naked mice having a body weight of 20-22 g were again used as animal models, each having a subcutaneously transplanted tumor of type “human bladder cancer RT4”. Six mice each constituted a test group. The mice of the test group A served for control. They were neither irradiated nor injected with a substance. The mice of the test group B were injected with 15 mg photofrin per kg body weight, without additionally irradiating them. The mice of the test groups C, D, E, F and G were each injected with 15 mg, 10 mg, 7,5 mg, 5 mg and 2,5 mg photofrin per kg body weight. After 24 h, an irradiation with 5 Gy followed. The tumor size was daily measured. The results of these measurements are listed in table 3.

TABLE 3
Tumor size (mm³)

| Day | A | B | C | D | E | F | G |
|---|---|---|---|---|---|---|---|
| 0 | 2.9 | 2.86 | 2.96 | 2.91 | 2.86 | 2.85 | 2.92 |
| 1 | 3.0 | 2.9 | 2.99 | 2.98 | 2.93 | 2.97 | 2.98 |
| 2 | 3.18 | 3.05 | 3.04 | 3.01 | 3.0 | 3.17 | 3.21 |
| 3 | 3.81 | 3.38 | 3.17 | 3.12 | 3.1 | 3.41 | 3.58 |
| 4 | 4.64 | 3.87 | 3.01 | 3.27 | 2.93 | 3.74 | 4.05 |
| 5 | 4.8 | 3.96 | 3.49 | 3.5 | 3.5 | 3.9 | 4.72 |
| 6 | 5.97 | 5.31 | 3.96 | 3.34 | 3.77 | 4.32 | 5.59 |
| 7 | 6.97 | 6.42 | 3.96 | 4.0 | 4.07 | 4.72 | 6.65 |
| 8 | 7.17 | 7.65 | 4.3 | 4.37 | 4.4 | 5.24 | 7.92 |
| 9 | 9.54 | 8.83 | 4.7 | 4.83 | 4.43 | 5.87 | 9.17 |
| 10 | 11.24 | 10.55 | 5.18 | 5.28 | 5.22 | 6.55 | 10.81 |
| 11 | 13.1 | 12.63 | 5.85 | 5.74 | 5.73 | 7.65 | 11.31 |

### Explanation of Symbols

A=control, not injected and not irradiated mice

B=mice injected with 15 mg photofrin per kg body weight

C=mice injected with photofrin (15 mg/kg), and irradiated with 5 Gy

D=mice injected with photofrin (10 mg/kg), and irradiated (5 Gy)

E=mice injected with photofrin (7,5 mg/kg), and irradiated (5 Gy)

F=mice injected with photofrin (5 mg/kg), and irradiated (5 Gy)

G=mice injected with photofrin (2,5 mg/kg), and irradiated (5 Gy)

In summary, it is to be stated that photofrin, especially in a dose of 7,5 to 15 mg per kg body weight, acts very well as a radiosensitizer. In this dose range, no significant differences with respect to the effectiveness can be detected. If photofrin is used in a lower dose (2,5-5 mg/kg body weight), it also acts as a radiosensitizer—but with less success. With the here tested high dosages photofrin also has no radioprotective effect.

Photofrin can be used as a radiosensitizer up to a dose of 20-30 mg per kg body weight. A higher dose would act toxically. Additionally, also higher radiation doses can be used. Suitable doses are in the range of 2 to 20 Gy, better 5 to 15 Gy, especially at 10 Gy.

1. Use of photofrin as a radiosensitizer in the treatment of tumorous tissue, wherein after a predetermined time interval after application of the photofrin an irradiation of the tumorous tissue with ionizing radiation is effected.

2. Use according to claim 1,
characterized in that
the photofrin is labeled with a neutron absorber.

3. Use according to claim 2,
characterized in that
the neutron absorber is boron and/or gadolinium.

4. Use according to claim 1,
characterized in that
the ionizing radiation is photon and/or electron and/or gamma and/or alpha and/or beta and/or neutron and/or proton radiation.

5. Use according to claim 1,
characterized in that
the irradiation is effected with a radiation dose of 2 to 20 Gy, more particularly 5 to 15 Gy.

6. Use according to claim 1,
characterized in that
the irradiation is fractionating irradiation.

7. Use according to claim 1,
characterized in that
the application of the photofrin is performed intravenously.

8. Use according to claim 7,
characterized in that
photofrin is injected intravenously in a dose of 7,5 mg to 15 mg per kg body weight.

9. Use according to claim 1,
characterized in that
the time interval is between 12 h and 48 h, more particularly at 24 h.

10. Use according to claim 1,
characterized in that
photofrin is a component of a medicine for treatment of tumorous tissue.
